(19) United States
(12) Patent Application Publication
(10) Pub. No.: US 2024/0341958 A1
(43) Pub. Date: Oct. 17, 2024

(54) DEVICES, SYSTEMS, AND METHODS FOR INTERNAL BANDING FOR REDUCTION OF VENOUS REFLUX

(71) Applicant: Kar Health, LLC, Iowa City, IA (US)

(72) Inventors: Wassef Karrowni, Cedar Rapids, IA (US); Mirna Karouni, Cedar Rapids, IA (US)

(21) Appl. No.: 18/629,738

(22) Filed: Apr. 8, 2024

Related U.S. Application Data
(60) Provisional application No. 63/457,971, filed on Apr. 7, 2023.

(51) Int. Cl. A61F 2/24

(52) U.S. Cl. CPC A61F 2/2475; A61F 2220/0016; A61F 2230/001; A61F 2230/0039; A61F 2230/0093; A61F 2250/0039; A61F 2250/0076

(57) ABSTRACT

A method and associated device for constricting fluid flow in vasculature comprising inserting a device into vasculature of a patient. The device comprising a frame defining an inflow portion, an outflow portion, and a center portion between the inflow portion and outflow portion and a fluid impermeable membrane lining at least the center portion, wherein a minimum diameter of the center portion is at least about 33% less than a diameter of the inflow portion, and the patient has deep vein reflux (DVR), chronic venous insufficiency (CVI), or excess shunting through a fistula. The method may further include placing the device within a vein proximally to a refluxing deep vein in a valve free zone.

### CROSS-REFERENCE TO RELATED APPLICATION(S)

This application claims the benefit under 35 U.S.C. § 119(e) to U.S. Provisional Application 63/457,971, filed Apr. 7, 2023, and entitled “Devices, Systems, And Methods For Internal Banding For Reduction Of Venous Reflux”, which is hereby incorporated herein by reference in its entirety for all purposes.

### TECHNICAL FIELD

The disclosure relates to devices, methods, and related systems for treating venous reflux, excess shunting through an arteriovenus fistula, and related conditions.

### BACKGROUND

Chronic venous insufficiency (CVI) is a common condition that occurs when the leg veins do not function normally. As would be understood, veins in the legs have valves that function as one-way doors directing blood flow toward the heart. When these valves do not work well-valve incompetence-blood can flow backwards, away from the heart, causing blood to pool in the legs and putting increased pressure on the walls of the veins. CVI is not a benign condition and can lead to co-morbidities and disability, including, for example, leg pain, edema, bulging veins, and skin discoloration/damage. In more serious cases leg ulcerations can occur which may be difficult to heal and tend to recur frequently.

CVI can develop from valve incompetence in the superficial veins, deep veins, and/or the perforating veins. There are well established and effective treatment options for superficial and perforator vein insufficiency. However, there is a lack of treatment options for CVI in the deep veins. As would be understood, in a significant proportion of patients treating superficial and perforating vein without addressing the deep vein reflux (DVR) does not provide enough clinical benefit, especially in regard to leg ulcer healing and preventing recurrence.

As would be understood, DVR results from three different etiologies: primary deep valve incompetence where the valve cusps are present but malfunctioning; secondary to deep vein thrombosis (post-thrombotic syndrome) where the valve cusps become fibrotic and immobile; and congenital valve malformation. As would be appreciated by those of skill in the art, there are multiple surgical techniques offered to patients with DVR. These surgical options include valvuloplasty (internal or external), transposition of femoral vein, vein transplant, neovalve creation, and/or external valve banding. These surgical techniques require high level surgical skills and expertise, and tend to be associated with complications including infection, nerve injury, arterial injury, painful and prolonged recovery, and deep vein thrombosis. One such surgical technique, known in the art, is Popliteal Vein External Banding (PVEB). Even though PVEB is described in the medical literature as an effective method in treating patients with DVR, it is an invasive surgical procedure and has limitations. Thus, there is large unmet need for less invasive and easily reproducible ways of treating DVR.

### BRIEF SUMMARY

Disclosed herein are various devices for correcting valvular insufficiency and/or reflux comprising a stent frame comprising an inflow portion, a center portion, and an outflow portion, wherein the inflow portion and outflow portion have a diameter greater than a diameter of the center portion. Various further implementations may be used to reduce blood flow in cases of excess shunting through a fistula, such as an arteriovenous fistula (AVF).

In Example 1, a device for correcting valvular insufficiency and reflux, comprising a stent frame comprising an inflow portion, a center portion, and an outflow portion, wherein the inflow portion and outflow portion have a diameter greater than a diameter of the center portion.

Example 2 relates to the device of any of Examples 1 and 3-9, further comprising one or more barbs on the inflow portion and outflow portion configured to contact a vein wall.

Example 3 relates to the device of any of Examples 1-2 and 4-9, further comprising an impermeable membrane along the center portion and at least parts of the inflow and outflow portions.

Example 4 relates to the device of any of Examples 1-3 and 5-9, wherein the device has a compressed configuration and an expanded configuration and wherein the device in the compressed configuration during insertion and is in the expanded configuration when placed within a vein.

Example 5 relates to the device of any of Examples 1-4 and 6-9, wherein the center portion has a minimum diameter at least about 20% less than the diameter of the inflow portion.

Example 6 relates to the device of any of Examples 1-5 and 7-9, wherein the middle portion has a generally symmetric shape.

Example 7 relates to the device of any of Examples 1-6 and 8-9, wherein the middle portion has a generally asymmetric shape.

Example 8 relates to the device of any of Examples 1-7 and 9, wherein the middle portion further comprises a tapered section and an orifice plate or sinus.

Example 9 relates to the device of any of Examples 1-8, wherein the middle portion is asymmetrically constricted thereby increasing resistance to reflux when compared to normal flow through the device.

In Example 10, a device for constricting fluid flow in vasculature comprising a frame defining an inflow portion, an outflow portion, and a center portion between the inflow portion and outflow portion, an fluid impermeable membrane lining at least the center portion, and at least one barb disposed on the frame configured to engage a vascular wall, wherein a minimum diameter of the center portion is at least about 33% less than a diameter of the inflow portion.

Example 11 relates to the device of any of Examples 10 and 12-18, wherein the support frame is a rigid or semirigid metal frame.

Example 12 relates to the device of any of Examples 10-11 and 13-18, wherein the center portion has a minimum diameter at least about 50% less than the diameter of the inflow portion.

Example 13 relates to the device of any of Examples 10-12 and 14-18, wherein the center portion has a generally asymmetric funnel shape creating differential flow pattern where normal flow is more efficient than reflux flow.

Example 14 relates to the device of any of Examples 10-13 and 15-18, wherein the center portion further comprises a tapered section and an orifice plate.

Example 15 relates to the device of any of Examples 10-14 and 16-18, wherein the center portion further comprises a tapered section and at least one sinus.

Example 16 relates to the device of any of Examples 10-15 and 17-18, wherein the center portion has a generally symmetric hourglass shape comprising two tapered sections.

Example 17 relates to the device of any of Examples 10-16 and 18, wherein the device is comprised of a semi flexible material allowing the device to be compressed for insertion.

Example 18 relates to the device of any of Examples 10-17, wherein the inflow portion, center portion, and outflow portion are unitary.

In Example 19, a method for constricting fluid flow in vasculature comprising inserting a device into vasculature of a patient. The device comprising a frame defining an inflow portion, an outflow portion, and a center portion between the inflow portion and outflow portion, and a fluid impermeable membrane lining at least the center portion, wherein a minimum diameter of the center portion is at least about 33% less than a diameter of the inflow portion, and the patient has deep vein reflux (DVR), chronic venous insufficiency (CVI), or excess shunting through a fistula.

Example 20 relates to the method of Examples 19, further comprising placing the device within a vein proximally to a refluxing deep vein in a valve free zone.

In Example 21 a method for treating deep vein reflux (DVR), chronic venous insufficiency (CVI), excess shunting through a fistula, or related conditions comprising inserting the device of any of claims 1-20 into a patient.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a depiction of a device with an hourglass center portion, according to one implementation.

FIG. 2 is a depiction of a device with a center portion having a funnel and sinuses, according to one implementation.

FIG. 3 is a depiction of a device with an hourglass center portion and membrane extending along the length of the device, according to one implementation.

FIG. 4 is a depiction of a device with a circular band within the center portion, a membrane extending along the length of the device, and a frame having a diamond pattern, according to one implementation.

FIG. 5 is a depiction of a device with a circular band within the center portion, a membrane extending along the length of the device, and a frame having a wave pattern, according to one implementation.

FIG. 6 is a depiction of a device with a funnel and sinuses within the center portion, a membrane extending along the length of the device, and a frame having a wave pattern, according to one implementation.

FIG. 7 shows a device having a funnel shaped center portion, a membrane along the center portion, and a frame having a diamond pattern, in place within vasculature, according to one implementation.

FIG. 8 shows a device having a center portion with a circular band, a membrane along the center portion, and a frame having a diamond pattern, in place within vasculature, according to one implementation.

FIG. 9 shows a device having a funnel shaped center portion with sinuses, a membrane along the center portion, and a frame having a diamond pattern, in place within vasculature, according to one implementation.

FIG. 10 shows a device having a center portion with a circular band, a membrane along the length of the device, and a frame having a wave pattern, in place within vasculature, according to one implementation.

FIG. 11 shows a device having a funnel shaped center portion with sinuses, a membrane along the length of the device, and a frame having a wave pattern, in place within vasculature, according to one implementation.

FIG. 12 shows a device having a funnel shaped center portion, a membrane along the center portion, and a frame having a diamond pattern, in place within vasculature, according to one implementation.

### DETAILED DESCRIPTION

Described herein is a device for minimally invasive treatment of DVR, CVI, excess shunting through a fistula, and other related diseases/syndromes/conditions. In various implementations, the device creates an internal banding inside a short segment of a vein proximal to a refluxing valve, optionally a deep vein proximal to a deep refluxing valve. In various implementations, the device allows for the use of minimally invasive surgical techniques for implantation/placement, including the use of wires and catheters, to create results similar to or better than known invasive procedures while avoiding the many complications of an invasive procedure.

As would be understood, during PVEB, an incision is made in the popliteal fossa, and the popliteal vein is exposed and separated. The banding segment is routinely selected to be proximal to the entries of greater vessels, in a valve free zone, and proximal to the popliteal valve. The circumference of the popliteal vein is exteriorly measured, and a polyester-urethane vascular patch is wrapped and sutured around the popliteal vein to reduce its original circumference by one-third. As would be understood, the one-third reduction of the original popliteal vein diameter is based on the fact that when a normally functioning deep vein valve is fully open, the cross-sectional area between the leaflets is 35% smaller than that of the vein distal to the valve. The PVEB technique is intended to simulate the anatomic shape of a normal vein and to modify deep vein valve reconstruction surgery in a valve-independent fashion.

In practice, the PVEB technique has been found to improve venous return flow without causing distal venous hypertension in the peritoneal vein or tibial veins. From a physiologic standpoint, banding of a segment of the vein proximal to a refluxing valve creates a localized pressure gradient at the popliteal vein that improves venous return, reducing reflux, and promotes venous ulcer healing. Analysis of patients pre- and post-surgery confirmed significant increases in the pressure gradient in the popliteal segment after PVEB (both in resting and Valsalva conditions). PVEB has also showed reduced reflux time and volume. Post-procedural duplex ultrasound and venography has shown competence of PVEB. Clinically, PVEB can achieve short ulcer healing time, and low long-term ulcer recurrence rate.

Yet, PVEB is a complex surgical procedure that requires high level of expertise and surgical skill. AS such, it is currently available only at a limited number of centers in the world. Given its invasive nature, it is also limited by other complications including prolonged hospital stays, deep vein thrombosis (DVT), discomfort at the incision site, incision site infection, bleeding, etc.

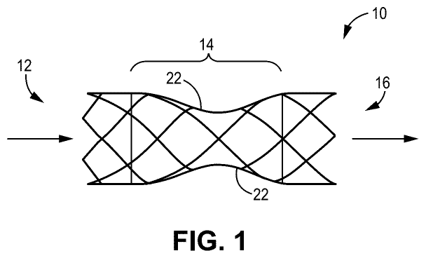

Disclosed herein is a device for minimally invasive treatment of DVR, valve insufficiency, reflux, excess shunting through an arteriovenous fistula, and other related diseases/syndromes/conditions. Turning to the figures in further detail, the device 10 includes a stent frame having three or more sections, an inflow 12, a center 14, and an outflow 16 section, as seen in FIGS. 1-6.

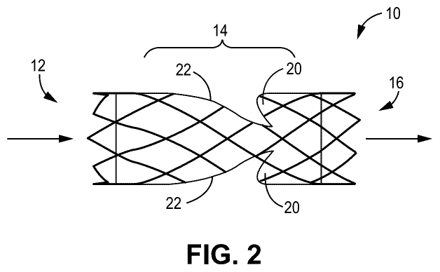

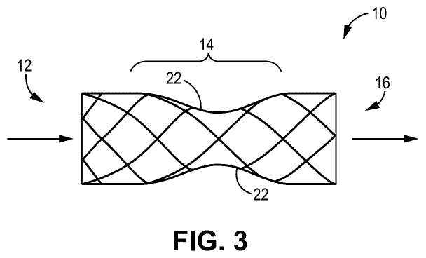

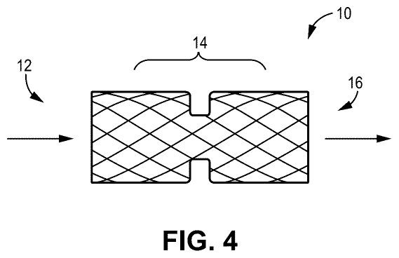

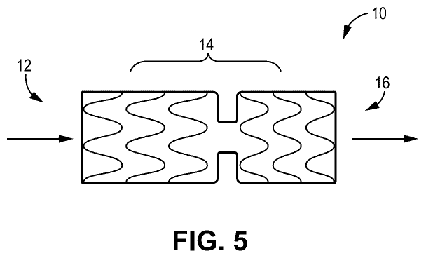

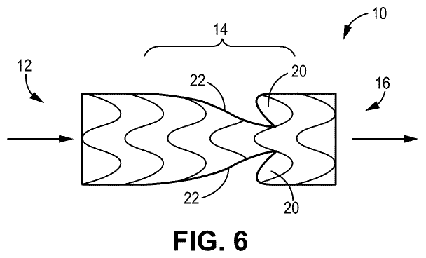

In various implementations, the device 10 may have a substantially hourglass shape with the inflow 12 and outflow 16 sections having a greater diameter than the center 14 section. The middle 14 section of the device 10 may have various shapes, as seen various in FIGS. 1-6, such as, but not limited to, an hourglass (FIGS. 1 and 3), funnel (FIGS. 2 and 6), or circular band (FIGS. 4 and 5). As can be seen, the center 14 section is shaped and arranged to constrict the vein, and therefore flow, to decrease flow through the vein, such as reflux flow.

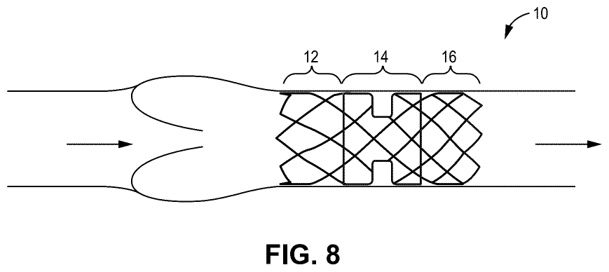

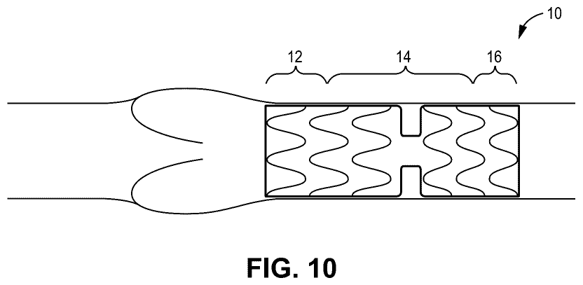

In certain implementations, the center 14 section provides narrowing with a symmetric shape toward the inflow 12 and outflow 16 ends, such as seen in FIGS. 1, 3, 4, 5, 8, and 10. In certain of these implementations, the center 14 section has two sloped/tapered sections 22 towards the inflow 12 and outflow 16 sections. Alternatively, the center 14 section may include a step or other substantially perpendicular protrusion into the lumen of the device 10 creating a circular band with or without a tapered portion. In these implementations, the center 14 section constricts forward and backward flow in a symmetric fashion.

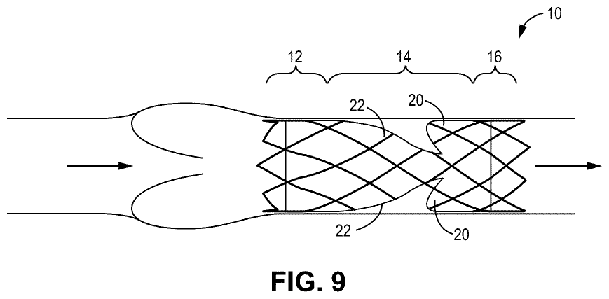

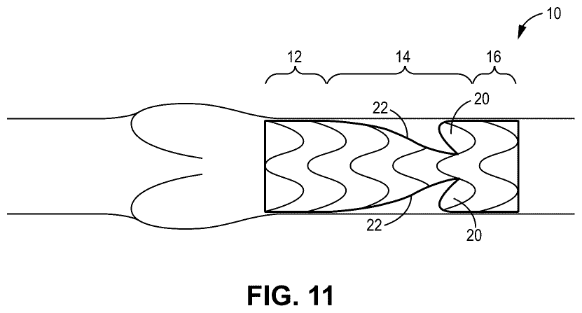

Alternatively, the center 14 section may have an asymmetric shape, as shown for example in FIGS. 2, 6, 7, 9, 11, and 12, increasing resistance to flow from the outflow 16 to inflow 12 portions of the device 10 (reflux) when compared to resistance to flow from the inflow 12 to outflow 16 portions (normal flow). That is, increased resistance to reflux while providing less restriction to normal flow. In these implementation, the device may include one tapered/sloped 22 section (forming a funnel shape) and optional sinuses 20 or plate 18 at the proximal end of the device 10.

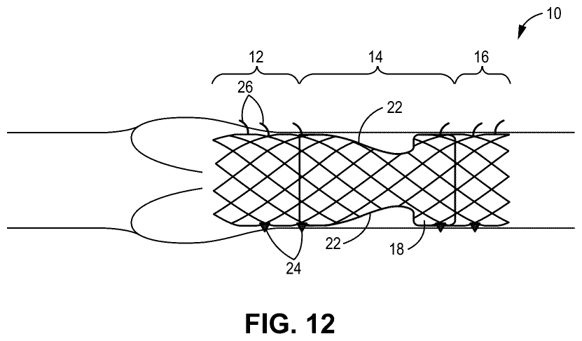

In implementations having an asymmetric shape of the center 14 section, the device 10 creates a differential in flow pattern where the forward flow (from inflow 12 to outflow 16) is more efficient (through a funnel shape or convergent nozzle) than the backward flow/reflux (from outflow 16 to inflow 12). The device 10 may include an orifice plate 18 (shown in FIG. 12) or other less flow efficient shapes for example sinuses 20 or grooves 20 (shown in FIGS. 2, 6, 9, and 11), to restrict the backward flow/reflux. The sinuses 20 or orifice plate 18 are configured to create a less flow efficient surface that limits backward flow (or reflux), in comparison to a tapered surface 22 that favors flow.

In various implementations, the device 10 is self-expandable, having a compressed configuration and an expanded configuration. As would be understood, the device 10 may be in a compressed configuration to assist with delivery, optionally via a catheter, and may be in an expanded configuration once deployed within the vein. That is, the device 10 may bend or otherwise deform to create of smaller effective circumference for insertion and transport to the desired location with the vasculature. When the device 10 reaches the desired location the device 10 may expand to its normal or expanded configured to fill the space within the vasculature. The device 10 may be oversized for the vein to be held in place. Additionally or alternatively, the device 10 may include one or more barbs, hooks, etc. (described further below) for attachment of the device to the vascular wall.

In various implementations, the device 10 may be made of memory shaped material, such as nitinol, stainless steel (316L), cobalt-chromium (Co—Cr) alloy (e.g. Elgiloy), or the like as would be understood. In various implementations, the device 10 is unitary, with each portion made from the same material. That is, the center 14 section is part of a single frame forming the inflow 12 and outflow 14 sections of the device.

In some implementations, the center section 14 of the device 10 is coated with an impermeable membrane sealing the device 10 such that fluid flows only through the center lumen of the device 10, and not outside of the center 14 portion of the device. The membrane may be composed of polytetrafluoroethylene (PTFE), polyethylene terephthalate (PET), bovine pericardium, porcine pericardium, or other similar material or materials as would be appreciated.

In certain implementations, the membrane may extend beyond the center 14 section onto the inflow 12 and/or outflow 16 sections or portion(s) thereof, as would be appreciated.

In alternative implementations, the device 10 is composed of a flexible membrane or graft, to act as a conduit for blood circulation. The flexible membrane may be polytetrafluoroethylene (PTFE), polyethylene terephthalate (PET/polyester/Dacron), or other similar material(s), as would be appreciated.

In these and other implementations, a supporting frame (optionally rigid or semirigid) may be connected to the flexible membrane (graft). The frame may have various configurations including continuous patterns (e.g. Z-shapes or diamond shapes), interrupted rings, helical shapes, or alternative shapes/patterns as would be appreciated. In various implementations, the frame may be formed from stainless steel wire, cobalt chromium (Co—Cr) alloy, nitinol, and/or other similar material, as would be appreciated. Additionally, the frame may include hooks, barbs, or other connecting components for fixational of the flexible membrane (graft) to the vein wall.

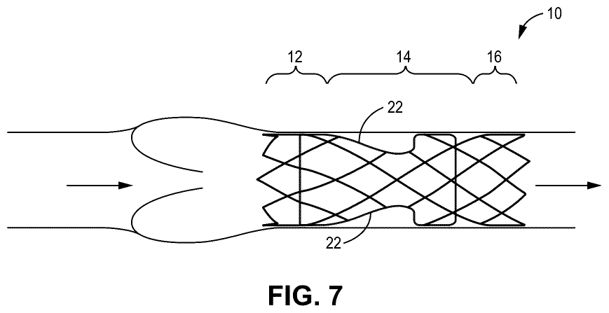

As can be seen in FIGS. 7-12, when in place the device 10 contacts the vein at the inflow 12 and outflow 16 sections. In certain implementations, the center 14 section of the device 10 does not contact the vein wall, or contact is minimized to only a portion of the center 14 section.

In certain implementations, the inflow 12 and outflow 16 portions of the device 10 are oversized for the vein size to insure contact of these portions with the vein wall and stability when in the intended position. In various alternative implementations, shown in FIG. 12, the device 10 may be fixed to the vein wall by anchors, hooks 26, barbs 24, or similar components extending from the outflow and/or inflow portions of the device/frame. These anchors, hooks 26, and/or barbs 24 may imbed into the vein wall after deployment of fix the device 10 in place within the vein.

In various implementations, for delivery, the device 10 is in a compressed configuration and delivered through a catheter or sheath and over a guide wire. Introduction of the device 10 into a vein via a catheter or sheath can be done via standard techniques known to those of skill in the art. Placement of the device 10 can be assisted by using contrast venography, intravascular ultrasound, extravascular ultrasound, or similar technique(s), as would be understood.

In various implementations, the device 10 is placed within a vein proximally to a refluxing deep vein in a valve free zone. The device 10 may be placed in various locations including the popliteal vein, femoral vein, common femoral vein, iliac vein, etc.

Alternatively, the device 10 may be used to reduce flow in cases of excess shunting through a arteriovenous fistula (AVF). In these implementations, the device 10 may be place in the outflow limb of the AVF. As would be understood AVF can result in dialysis-access steal syndrome.

As would be appreciated, the diameter/size of the device 10 (stent or graft style) is dependent on the patient and selected location. The size of the device 10 can be determined by imaging, prior to and/or during the placement procedure. In various implementations, the inflow 12 and outflow 14 section diameter may range from about 10 mm to about 20 mm. The diameter of the center section may vary to be about 20% to about 50% of the inflow 12/outflow 16 diameter. Other sizes and ranges are a possible and would be appreciated by those of skill in the art.

Although the disclosure has been described with references to various embodiments, persons skilled in the art will recognize that changes may be made in form and detail without departing from the spirit and scope of this disclosure.

What is claimed is:

1. A device for correcting valvular insufficiency and reflux, comprising a stent frame comprising an inflow portion, a center portion, and an outflow portion, wherein the inflow portion and outflow portion have a diameter greater than a diameter of the center portion.

2. The device of claim 1, further comprising one or more barbs on the inflow portion and outflow portion configured to contact a vein wall.

3. The device of claim 1, further comprising an impermeable membrane along the center portion and at least parts of the inflow and outflow portions.

4. The device of claim 1, wherein the device has a compressed configuration and an expanded configuration and wherein the device in the compressed configuration during insertion and is in the expanded configuration when placed within a vein.

5. The device of claim 1, wherein the center portion has a minimum diameter at least about 20% less than the diameter of the inflow portion.

6. The device of claim 1, wherein the middle portion has a generally symmetric shape.

7. The device of claim 1, wherein the middle portion has a generally asymmetric shape.

8. The device of claim 7, wherein the middle portion further comprises a tapered section and an orifice plate or sinus.

9. The device of claim 1, wherein the middle portion is asymmetrically constricted thereby increasing resistance to reflux when compared to normal flow through the device.

10. A device for constricting fluid flow in vasculature comprising:
(a) a frame defining an inflow portion, an outflow portion, and a center portion between the inflow portion and outflow portion;
(b) an fluid impermeable membrane lining at least the center portion; and
(c) at least one barb disposed on the frame configured to engage a vascular wall,
wherein a minimum diameter of the center portion is at least about 33% less than a diameter of the inflow portion.

11. The device of claim 10, wherein the support frame is a rigid or semirigid metal frame.

12. The device of claim 10, wherein the center portion has a minimum diameter at least about 50% less than the diameter of the inflow portion.

13. The device of claim 10, wherein the center portion has a generally asymmetric funnel shape creating differential flow pattern where normal flow is more efficient than reflux flow.

14. The device of claim 13 wherein the center portion further comprises a tapered section and an orifice plate.

15. The device of claim 13, wherein the center portion further comprises a tapered section and at least one sinus.

16. The device of claim 10, wherein the center portion has a generally symmetric hourglass shape comprising two tapered sections.

17. The device of claim 10, wherein the device is comprised of a semi flexible material allowing the device to be compressed for insertion.

18. The device of claim 10, wherein the inflow portion, center portion, and outflow portion are unitary.

19. A method for constricting fluid flow in vasculature comprising:
inserting a device into vasculature of a patient, the device comprising:
(a) a frame defining an inflow portion, an outflow portion, and a center portion between the inflow portion and outflow portion; and
(b) a fluid impermeable membrane lining at least the center portion,
wherein a minimum diameter of the center portion is at least about 33% less than a diameter of the inflow portion, and the patient has deep vein reflux (DVR), chronic venous insufficiency (CVI), or excess shunting through a fistula.

20. The method of claim 19, further comprising placing the device within a vein proximally to a refluxing deep vein in a valve free zone.
